(19) United States
(12) Patent Application Publication
(10) Pub. No.: US 2003/0039672 A1
(43) Pub. Date: Feb. 27, 2003

(54) Cosmetic composition and method of treating skin

(75) Inventors: Rebecca Susan Ginger, Bedford (GB); Andrew Easson Mayes, Bedford (GB); Julia Sarah Rogers, Bedford (GB); Paula Rachel Yates, Bedford (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/211,427

(22) Filed: Aug. 6, 2002

(30) Foreign Application Priority Data
Aug. 10, 2001 (GB) 0119583.3

(51) Int. Cl. A61K 7/00; A61K 31/7024; A61K 31/202

(52) U.S. Cl. 424/401; 514/25; 514/560

(57) ABSTRACT

A cosmetic method for treating aged, sensitive, dry, flaky, wrinkled and/or photodamaged skin is provided through topical application of a composition which comprises an unsaturated C16 fatty acid having at least three double bonds, which may be preferably hexadecatrienoic acid, and/or derivatives thereof. The invention also relates to compositions suitable for such cosmetic treatment

This invention relates to a cosmetic compositions, and to cosmetic methods of improving the condition and appearance of skin involving the use of highly unsaturated C16 fatty acids having at least three double bonds, and especially hexadecatrienoic acid. The invention also relates to the preparation of topical compositions for improving the condition and appearance of skin.

Skin is subject to deterioration through dermatological disorders, environmental abuse (wind, air conditioning, central heating) or through the normal aging process (chronoaging) which may be accelerated by exposure of skin to sun (photoaging). In recent years the demand for cosmetic compositions and cosmetic methods for improving the appearance and condition of skin has grown enormously.

Consumers are increasingly seeking “anti-aging” cosmetic products which treat or delay the visible signs of chronoaging and photoaging skin such as wrinkles, lines, sagging, hyperpigmentation and age spots.

Consumers also frequently seek other benefits from cosmetic products in addition to anti-aging. The concept of “sensitive skin” has also raised the consumer demand for cosmetic products which improve the appearance and condition of sensitive, dry and/or flaky skin and to soothe red, and/or irritated skin. Consumers also desire cosmetic products which treat spots, pimples, blemishes etc.

Many people are concerned with the degree of pigmentation of their skin. For example, people with age spots or freckles may wish such pigmented spots to be less pronounced. Others may wish to reduce the skin darkening caused by exposure to sunlight or to lighten their natural skin colour. To meet this need many attempts have been made to develop products that reduce the pigment production in the melanocytes. However, the substances thus far identified tend to have undesirable side effects, e.g. skin irritation.

Consequently such substances are not suitable for cosmetic use, or they can only be applied at a concentration at which their skin lightening effect is less than desired. Using a combination of different skin lightening substances may be considered to reduce adverse side effects but there is a substantial risk that by using such a combination the skin lightening is reduced as well due to competition effects. Therefore there is a need for improvement in the effectiveness of cosmetic skin lightening products particularly, such that they do not irritate the skin.

In its broadest aspect, we have found that the use of highly unsaturated C16 fatty acids, which preferably comprise at least three double bonds, and may comprise four or five double bonds, may be beneficial in providing a topical composition with skin care benefits.

We have now found that effective treatment and prevention of normal skin conditions due to chronoaging or photoaging, such as wrinkles, lines, sagging, hyperpigmentation and age spots, may be obtained through the application of cosmetic compositions to the skin which comprise highly unsaturated C16 fatty acids comprising at least three double bands, or derivatives thereof. We have also found that the use of such unsaturated C16 fatty acids, which in a preferred aspect may comprise three double bonds, in cosmetic compositions advantageously may provide further skin benefits in addition to anti-aging, such as for soothing sensitive and/or irritated skin, improved resilience and reduced dryness/flakiness, and lightening the skin.

Thus, according to a first aspect of the invention, there is provided a topical composition for application to the human skin comprising an effective amount of a highly unsaturated C16 fatty acid having at least three double bonds, and derivatives thereof.

Suitable highly unsaturated fatty acids for use according to the invention are C16 fatty acids which have at least three double bonds, and may have four or five double bonds.

A preferred C16:3 fatty acid is hexadecatrienoic acid (C16:3(c7, c10, c13)), which is also known as hiragonic acid. This acid is known to occur in photosynthetic leaves, such as for example rape leaves, fern lipid, ginko leaves, potato leaves, tomato leaves and spinach. It may also occur in the leaves of Brassicaceae plants, such as horse radish, cabbage, turnip, Chinese mustard, cauliflower and watercress.

Other suitable highly unsaturated C16 fatty acids include: Hexadecatrienoic acid (C16:3(c6, c9, c12)). This is obtainable from micro algae Skeletonema Costatum (Virron et al; Analytica Chimica Acta 409(200),257-266);

Hexadecatetraenoic acid (C16:4(c4, c7, c10, c13)). This material is obtainable from Australian Marine Sponge, Callyspongia sp. (Urban and Capan, Lipids 32 (1997), 6,675-77);

10-hydroxy Hexadecatrienoic acid (c7, t11, c13). This is obtainable from Lemna Minor (duckweed). (Previtere and Monaco, Phytochemistry 22 (1983), 1445-1446);

7-hydroxy Hexadecatrienoic acid (t8, c10, c13). Obtainable from Elodea Canodensis (Previtere et al, Phytochemistry 24 (1985), 1838-1840). This material may also be a source of 10-hydroxy hexadecatrienoic acid (see above);

Hexadecatetraenoic acid (C16:4(c6, c9, c12, c15)). This is obtainable from Antartic Sea ice diatom, Stauroneis amphioxys, (Gillin et al, Phytochemistry 20 (1981), 1935-37). This material may also be a source of C16:3(c6, c9, C12); Hexadecapentaenioic acid (C16:5(c4, c7, t9, t11, c13)). This may be found in the marine green microalga Anadyomene stellata (Mikhailova M V et al, Lipids 1995, 30, 583).

Marine phytoplanktons (Cylindrotheca closterium and Gymnodinium mikimotoi) are also described as containing suitable C16:3 unsaturated fatty acids with a double bond in the 12 position (eg. C16:3(c6, c9, c12), Journal of the Japanese Society for Food Science and Technology, Nippon Shokuhin Kagaka Kogaku Kaishi 46:(1), 29-33, 1999).

Suitable C16:3 omega 3 fatty acids (eg. C16:3(c7, c10, c13)) may also be found in Codium sp. (green microalgae)(Phytochemistry 48:(8) 1335-1339, August 1998).

Free living nitrogen fixing cyanobacteria of the genera Anabaena and Nostoc are also known sources of C16:4 cis-4 and C16:3 cis-6 fatty acids (“Differentiation of Free living Anabaena and Mostoc Cyanobacteria on the basis of fatty acid composition”, Caudales R., Well J. M., International Journal of Systematic Bacteriology 42:(2)246-251, April 1992).

Preferred materials for use according to the invention are C16 fatty acids with three double bonds; a particularly preferred material for use in compositions according to the invention is hexadecatrienoic acid (C16:3(c7, c10, c13)).

According to a further aspect of the present invention there is provided a cosmetic method of providing at least one skin care benefit selected from: treating/preventing wrinkling, sagging, aged and/or photodamaged skin; boosting collagen deposition in skin, boosting decorin production in skin, enhancing tissue repair; lightening skin; improving skin condition and resilience through enhanced barrier formation; treating dry and flaky skin; soothing irritated, red and/or sensitive skin; and improving skin texture, smoothness and/or firmness; the method comprising applying to the skin a topical composition comprising a highly unsaturated C16 fatty acid and/or derivatives thereof.

The present invention also encompasses the use of a highly unsaturated C16 fatty acid and/or derivatives thereof in the preparation of a topical composition for providing at least one skin care benefit selected from treating/preventing wrinkling, sagging, aged and/or photodamaged skin; boosting collagen deposition in skin, boosting decorin production in skin, enhancing tissue repair; lightening skin; improving skin condition and resilience through enhanced barrier formation; treating dry and flaky skin; soothing irritated, red and/or sensitive skin; improving skin texture, smoothness and/or firmness.

The inventive methods and use of such highly unsaturated C16 fatty acids may thus provide anti-aging benefits which result in the promotion of smooth and supple skin with improved elasticity and a reduced or delayed appearance of wrinkles and aged skin, with improved skin colour. A general improvement in the appearance, texture and condition, in particular with respect to the radiance, clarity, and general youthful appearance of skin may be achieved. The inventive methods and uses are also beneficial for soothing and calming sensitive and/or irritated skin. The C16 highly unsaturated fatty acids may also useful for topical application to human skin for reducing melanin production and thus lightening the skin on which it has been applied. Thus the inventive methods advantageously provide a wide range of skin care benefits.

The term “treating” as used herein includes within its scope reducing, delaying and/or preventing the above mentioned skin conditions such as wrinkled, aged, photodamaged, and/or irritated skin and generally enhancing the quality of skin and improving its appearance and texture by preventing or reducing wrinkling and increasing flexibility, firmness, smoothness, suppleness and elasticity of the skin an skin lightening. The cosmetic methods and the uses of the unsaturated fatty acids and/or derivatives according to the invention may be useful for treating skin which is already in a wrinkled, aged, photo-damaged and irritated condition or for treating youthful skin to prevent or reduce those aforementioned deteriorative changes due to the normal aging/photoaging process.

The invention also includes derivatives, in particular monohydroxy derivatives of the free highly unsaturated (e.g. c16:3) fatty acids.

In one embodiment, unsaturated fatty acid moieties according to the invention conveniently have the double bonds at the 7, 10 and 13 positions. Preferable derivatives include those derived from substitution of the carboxyl group of the acid, such as esters (eg retinyl esters, triglyceride esters, monoglyceride esters, diglyceride esters, phosphoesters), amides (eg ceramide derivatives), salts (eg alkali metal and alkali earth metal salts, ammonium salts); and/or those derived from substitution of the C18 carbon chain, such as alpha hydroxy and/or beta hydroxy derivatives.

In the case of triglyceride ester derivatives, which when hydrolysed provide the highly unsaturated fatty acids which are the subject of the invention, all positional isomers of the unsaturated C16 fatty acid substituents on the glycerol backbone are included. The triglycerides must contain at least one highly unsaturated fatty acid moiety. For example, of the three esterifiable positions on the glycerol backbone, the 1 and 2 positions may be esterified with highly unsaturated C16 fatty acid and by another lipid at position 3 or as an alternative, the glycerol backbone could be esterified by the C16 fatty acid at the 1 and 3 positions with another lipid at position 2.

Oils that may be rich in the unsaturated acid triglyceride would thus also suitable for use in the present invention.

A suitable source of the highly unsaturated fatty acids are mono- and digalactosyl diglycerides, which in the broader form are classes of mono- and disaccharide esters of glycerol, which are found in a variety of plant sources. Wherever highly unsaturated fatty acids are referred to in this specification, it is to be understood that the derivatives thereof comprising highly unsaturated C16 fatty acid moieties are also included. “Highly unsaturated fatty acid moieties” refers to highly unsaturated fatty acyl portion(s) of a the fatty acid derivative.

The highly unsaturated fatty acid, to be employed in accordance with the present invention is present in the topical composition in an effective amount. Normally the total amount of the active is present in an amount between 0.0001% and 50% by weight of the composition. More preferably the amount is from 0.01% to 10% and most preferably from 0.1% to 5% in order to maximise benefits at a minimum cost.

The composition used according to the invention also comprises a dermatologically/cosmetically acceptable vehicle to act as a dilutant, dispersant or carrier for the active highly unsaturated fatty acid or its derivative. The vehicle may comprise materials commonly employed in skin care products such as water, liquid or solid emollients, silicone oils, emulsifiers, solvents, humectants, thickeners, powders, propellants and the like.

The vehicle will usually form from 5% to 99.9%, preferably from 25% to 80% by weight of the composition, and can, in the absence of other cosmetic adjuncts, form the balance of the composition.

Besides the highly unsaturated fatty acid active, other specific skin-benefit actives such as sunscreens, other skin lightening agents, and skin tanning agents may also be included. The vehicle may also further include adjuncts such as perfumes, opacifiers, preservatives, colourants and buffers. Other preferred adjuncts include other known skin care benefit agents, moisturisation agents, agents known to improve skin condition, and especially antioxidants, such as BHT, tocopherol, ascorbyl acetate, quercetins and green tea polyphenols.

To prepare the topical composition used in the method of the present invention, the usual manner for preparing skin care products may be employed. The active components are generally incorporated in a dermatologically acceptable carrier in conventional manner. The active components can suitably first be dissolved or dispersed-in a portion of the water or another solvent or liquid to be incorporated in the composition. The preferred compositions are oil-in-water or water-in-oil emulsions.

The composition may be in the form of conventional skin-care products such as a cream, gel or lotion or the like. The composition can also be in the form of a so-called “wash-off” product e.g. a bath or shower gel, possibly containing a delivery system for the actives to promote adherence to the skin during rinsing. Most preferably the product is a “leave-on” product; a product to be applied to the skin without a deliberate rinsing step soon after its application to the skin.

The composition may be packaged in any suitable manner such as in a jar, a bottle, tube, roll-ball, or the like, in the conventional manner.

The method of the present invention may be carried out one or more times daily to the skin which requires treatment. The improvement in skin appearance will usually become visible after 2 weeks to 6 months, depending on skin condition, the concentration of the active components used in the inventive method, the amount of composition used, the frequency with which it is applied, and the benefit being sought. In general, a small quantity of the composition, for example from 0.1 to 5 ml is applied to the skin from a suitable container or applicator and spread over and/or rubbed into the skin using the hands or fingers or a suitable device. A rinsing step may optionally follow depending on whether the composition is formulated as a “leave-on” or a “rinse-off” product.

In order that the present invention may be more readily understood, the following examples are given, by way of illustration only.

The invention will now be explained by way of example only.

### EXAMPLES

The following example demonstrates the anti-aging benefits of hexadecatrienoic acid (C16:3(c7, c10, c13)).

It is know from our co-pending European application No. 99908956.8 that topical retinoic acid treatments can be used to cause upregulation of procollagen I and decorin in vivo. To this end, the passages under the heading “Identification of procollagen I and decorin upregulation in skin in vivo following topical retinoic acid treatment for comparative purposes” in that application are incorporated herein in their entirety.

### Example 1

### Procedure For Measuring Procollagen-I and Decorin Synthesis In Human Dermal Fibroblasts

Preparation of Dermal Fibroblast Conditioned Medium

Primary human foreskin fibroblasts at passage 2 (P2) were seeded into 12-well plates at 10000 cells/cm² and maintained for 24 hours in an atmosphere of 5% carbon dioxide and 4% oxygen in Dulbeccos Modified Eagles Medium (DMEM) supplemented with 10% foetal calf serum. After this time the cells were washed with serum free DMEM and then incubated in fresh serum free DMEM for a further 60 hours. The fibroblast monolayers were then washed again with serum free DMEM. Test reagents and vehicle controls were added to the cells in triplicate in a final volume of 0.4 ml/well fresh serum free DMEM and incubated for a further 24 hours. This fibroblast conditioned medium was either analysed immediately or snap frozen in liquid nitrogen and stored at −70° C. for future analysis. The cells were then counted and data from the dot-blot analysis subsequently standardised to cell number.

Dot Blot Assay for Procollagen-I and Decorin Protein in Dermal Fibroblast Conditioned Medium

Samples of conditioned medium from dermal fibroblasts treated with vehicle (as a control) or test reagents were supplemented with 20 mM dithiothreitol (1:10 dilution of 200 mM stock solution) and 0.1% sodium dodecylsulphate (1:100 dilution of 10% stock solution), mixed well and then incubated at 75° C. for 2 minutes. A standard for the assay was generated by serial dilution of neat fibroblast conditioned medium from fibroblasts seeded at 10000 cells/cm² in a 175 cm² flask and maintained in serum free DMEM as described above.

Assay samples were subsequently applied in triplicate to a pre-wetted sheet of Immobilon-P transfer membrane using the 96-well Bio-Dot Apparatus from Bio-Rad as described in the manufacturers' guidelines. Approximately 200 μl of medium was applied per well. The medium was allowed to filter through the membrane under gravity (30 minutes) after which the membrane was washed twice with PBS (200 μl). These PBS washes were allowed to filter through the membrane under gravity (2×15 minutes). The Bio-Dot apparatus was then attached to a vacuum manifold and a third and final PBS wash carried out under suction. The apparatus was disassembled, the membrane removed and quickly cut as required before being placed in blocking buffer overnight at 4° C. Membranes prepared for decorin analysis were blocked with 3% (w/v) BSA/0.1% (v/v) Tween 20 in PBS, whilst those for procollagen-I analysis were blocked with 5% (w/v) non fat dried milk powder/0.05% Tween 20 in PBS. The following day, the membranes were probed with 1:10000 dilution of primary antibodies to either human procollagen-I (MAB1912; rat monoclonal; Chemicon Int. Inc., Temecula, Calif.) or human decorin (rabbit polyclonal; Biogenesis) for 2 hours at room temperature. The membranes were subsequently washed with TBS/0.05% Tween 20 (3×5 minutes) and then incubated with 1:1000 dilution of ¹²⁵I-conjugated anti-rat or anti-rabbit F(ab′)2 fragments (Amersham) as required for 1 hour at room temperature.

Following this the Immobilon strips were again washed with TBS/Tween 20 (3×5 minutes) before being allowed to dry in air at room temperature. The dried membranes were wrapped in cellophane and exposed to a Molecular Dynamics storage phosphor screen for 16-18 hours. At the end of this time the exposed screen was scanned by a phosphorimager (Molecular Dynamics Phosphorimager SF) using ImageQuant™ software. Dot intensity was assessed by computer-assisted image analysis using the quantification tools in ImageQuant™, standardised to cell number and the effects of various test reagents on decorin and procollagen-I synthesis were determined relative to a vehicle treated control value of 100 arbitrary units.

In order to normalise the results the effects of the test substance was determined relative to a vehicle treated control value of 100 arbitrary units. The results are shown numerically in Table 1, and indicate that hexadecatrienoic acid significantly upregulates the synthesis of both decorin and procollagen-I in human dermal fibroblasts as compared to the control.

TABLE 1
Decorin and Procollagen production

| Hexadecatrienoic acid | Procollagen | |
|---|---|---|
| Concentration | Level | Decorin Level |
| 1% Ethanol (0) | 100 | 100 |
| 1 μM | 91.0 | 108.7 |
| 10 μM | 184.2 | 115.4 |

### Example 2

Human foreskin keratinocytes at passage 3 (P3) were seeded into 96 well plates at 4000 cells/well in Dubleccos Modified Eagles Medium (DMEM), 0.03 mM calcium. The cells were grown for 3 days prior to treatment. The treatment vehicle was DMSO. After 4 days of treatment, the cells were harvested and washed three times with 100 μl phosphate buffered saline (PBS). The cells were then extracted in 1% Triton X100, 50 mM Tris pH 8.0, 0.02 mM Leupeptin, 0.02 mM Pepstatin. 60 il/well of extract was then assayed for DNA concentration (ng/well), Pico Green DNA assay, Molecular Probes.

The cells were then washed in 200 μl PBS, and then 100 μl of 2% SDS, 20 mM DTT was added to each well. The plates were then sealed with a Titertek plate sealer (ICN) and incubated at 60° C. over night in an air tight damp environment (i.e. a sealed sandwich box lined with damp paper). The extract was then filtered through a PVDF transfer membrane (Bio-rad) under gravity using Dot-Blot apparatus (Bio-rad). The membrane is then washed in distilled water prior to silver staining (Bio-rad Silver Stain kit). The stained dot blot membrane is then analysed using Phoretix array software (Phoretix International).

The results are shown in Tables 2 and 3.

TABLE 2
Cornified Envelope

| μM Hexadecatrienoic Acid | Mean | SD | % Control |
|---|---|---|---|
| 0 | 17995 | 8860 | — |
| 0.1 | 17617 | 3664 | 98 |
| 0.5 | 24009 | 9571 | 133 |
| 1 | 36017 | 15579 | 200 |
| 10 | 40997 | 8617 | 227 |

TABLE 3
DNA Results (ng/well)

| μM Hexadecatrienoic Acid | Mean | SD | % Control |
|---|---|---|---|
| 0 | 1.66 | 0.189 | — |
| 0.1 | 0.98 | 0.23 | 59 |
| 0.5 | 1.48 | 0.18 | 89 |
| 1 | 1.27 | 0.18 | 76 |
| 10 | 1.13 | 0.26 | 68 |

The results show how cornified envelope production increased in response to 1-10 μM application of hexadecatrienoic acid. This is indicative of enhanced keratinocyte differentiation, and suggests that hexadecatrienoic acid improves in situ skin barrier formation and resilience.

### Example 3

The formulation below describes an oil in water cream suitable for the methods and uses according to the present invention. The percentages indicated are by weight of the composition.

| | Wt % |
|---|---|
| Mineral Oil | 4 |
| Hexadecatrienoic acid | 1.15 |
| Brij 56* | 4 |
| Alfol 16RD** | 4 |
| Triethanolamine | 0.75 |
| Butane-1,3-diol | 3 |
| Xanthan gum | 0.3 |
| Perfume | Qs |
| Butylated hydroxy toluene | 0.01 |
| Water | to 100 |

*Brij 56 is cetyl alcohol POE (10)
**Alfol 16RD is cetyl alcohol

### Example 4

The formulation below describes an emulsion cream according to the present invention.

| FULL CHEMICAL | | |
|---|---|---|
| NAME OR CTFA NAME | TRADE NAME | WT. % |
| Hexadecatrienoic acid | | 2.0 |
| Disodium EDTA | Sequesterene Na2 | 0.05 |
| Magnesium aluminium | Veegum Ultra | 0.6 |
| silicate | | |
| Methyl paraben | Methyl Paraben | 0.15 |
| Simethicone | DC Antifoam Emulsion | 0.01 |
| Butylene glycol 1,3 | Butylene Glycol 1,3 | 3.0 |
| Hydroxyethylcellulose | Natrosol 250 HHR | 0.5 |
| Glycerine, USP | Glycerine USP | 2.0 |
| Xanthan gum | Keltrol 1000 | 0.2 |
| Triethanolamine | Triethanolamine (99%) | 1.2 |
| Stearic acid | Pristerene 4911 | 3.0 |
| Propyl paraben NF | Propylparaben NF | 0.1 |
| Glyceryl hydrostearate | Naturechem GMHS | 1.5 |
| Stearyl alcohol | Lanette 18 DEO | 1.5 |
| Isostearyl palmitate | Protachem ISP | 6.0 |
| C12-15 alcohols octanoate | Hetester FAO | 3.0 |
| Dimethicone | Silicone Fluid 200 | 1.0 |
| | (50 cts) | |
| Cholesterol NF | Cholesterol NF | 0.5 |
| Sorbitan stearate | Sorbitan Stearate | 1.0 |
| Butylated hydroxytoluene | Embanox BHT | 0.05 |
| Tocopheryl acetate | Vitamin E Acetate | 0.1 |
| PEG-100 stearate | Myrj 59 | 2.0 |
| Sodium stearoyl lactylate | Pationic SSL | 0.5 |
| Hydroxycaprylic acid | Hydroxycaprylic Acid | 0.1 |
| Retinyl palmitate | Vitamin A Palmitate | 0.06 |
| Alpha-bisabolol | Alpha-bisabolol | 0.2 |
| Water, DI | | q.s. |
| | | to 100 |

Both the above topical compositions of examples 3 and 4 provide a suitable cosmetic treatment which may improve the appearance of wrinkled, aged, photo-damaged, and/or irritated skin, when applied to skin that has deteriorated through the aging or photoaging or when applied to youthful skin to help prevent or delay such deteriorative changes. The compositions can be processed in conventional manner.,

1. A topical composition comprising:
(a) an effective amount of a highly unsaturated C16 fatty acid having at least three double bonds or derivatives thereof; and
(b) a dermatologically acceptable vehicle.

2. A cosmetic composition according to claim 1, wherein the fatty acid has three, four or five double bonds.

3. A cosmetic composition according to claim 2, wherein the fatty acid has three double bonds.

4. A cosmetic composition according claim 3, wherein the unsaturated C16 fatty acid has the double bond configuration (c7, c10, c13), (c6, c9, c12), (c7, t11, c13) or (t8, c10, c13).

5. A composition according to claim 4, wherein the unsaturated C16 fatty acid is hexadecatrienoic acid (C16:3(c7, c10, c13)).

6. A cosmetic composition according to claim 1, wherein the unsaturated C16 fatty acid is a hydroxy C16 unsaturated fatty acid.

7. A cosmetic composition according to claim 1, wherein the composition contains a mono- or disaccharide glycerin ester which is hydrolysable on the skin to provide the highly unsaturated c16 fatty acid having at least three double bonds.

8. A cosmetic composition according to claim 1 wherein the mono- or disaccharide glycerin ester is a mono- or digalactosyl glyceride.

9. A cosmetic method of providing at least one skin care benefit selected from: treating/preventing wrinkling, sagging, aged and/or photodamaged skin; boosting collagen deposition in skin, boosting decorin production in skin, enhancing tissue repair; lightening skin; improving skin condition and resilience through enhanced barrier formation; treating dry and flaky skin; soothing irritated, red and/or sensitive skin; and improving skin texture, smoothness and/or firmness; the method comprising applying to the skin a topical composition comprising an unsaturated C16 fatty acid having at least three double bonds, and/or derivatives thereof.

10. Use of an unsaturated C16 fatty acid having at least three double bonds, and/or derivatives thereof in the preparation of a topical composition for providing at least one skin care benefit selected from treating/preventing wrinkling, sagging, aged and/or photodamaged skin; boosting collagen deposition in skin, boosting decorin production in skin, enhancing tissue repair; lightenig skin; improving skin condition and resilience through enhanced barrier formation; treating dry and flaky skin; soothing irritated, red and/or sensitive skin; improving skin texture, smoothness and/or firmness.
